(19) United States
(12) Patent Application Publication
(10) Pub. No.: US 2026/0165971 A1
(43) Pub. Date: Jun. 18, 2026

(54) Single-Cell Microsphere Prepared Based on Oil-Free Aqueous Two-Phase System Strategy and Preparation Method Therefor

(71) Applicant: Guangzhou Medical University, Guangzhou (CN)

(72) Inventors: Guosheng TANG, Guangzhou (CN); Leyan XUAN, Guangzhou (CN); Maobin XIE, Guangzhou (CN); Xiyong YU, Guangzhou (CN); Yingying HOU, Guangzhou (CN); Jialin WU, Guangzhou (CN)

(21) Appl. No.: 19/533,769

(22) Filed: Feb. 9, 2026

Related U.S. Application Data
(63) Continuation of application No. PCT/CN2024/108647, filed on Jul. 30, 2024.

(30) Foreign Application Priority Data
Dec. 5, 2023 (CN) 202311652129.6

(51) Int. Cl. A61K 9/19; A61K 9/00; A61K 9/16

(52) U.S. Cl. CPC A61K 9/19; A61K 9/0019; A61K 9/0024; A61K 9/1652

(57) ABSTRACT

The present invention discloses a single-cell microsphere prepared based on an oil-free aqueous two-phase system strategy and a preparation method therefor. Specifically, through gravity induction, cells in an upper aqueous phase enter a lower aqueous phase; due to adhesion between the cells and hydrogel and a stable interface formed by two aqueous phases, the individual cells entering the lower aqueous phase are encapsulated by the hydrogel. Being free of oils and surfactants, the method ensures high cell viability and enables universal encapsulation across different cell types.

### CROSS-REFERENCE TO RELATED APPLICATIONS

The present application is a continuation of International Application No. PCT/CN2024/108647, filed on Jul. 30, 2024, which claims priority to Chinese Patent Application No. 202311652129.6, filed on Dec. 5, 2023, the entire disclosure of which is incorporated herein by reference.

### TECHNICAL FIELD

The present invention belongs to the technical fields of biomedicines, and particularly relates to a single-cell microsphere prepared based on an oil-free aqueous two-phase system strategy and a preparation method therefor.

### BACKGROUND

A main objective of cell therapy is to transplant cells into a patient's body to replace or repair damaged cells, thereby promoting regeneration and repair of tissues or organs. The cells (including stem cells) are typically implanted into damaged sites via direct injection; however, in many cases, direct cell transplantation is ineffective. First, due to shear forces between the cells in injection, mechanical pressures from receptor tissues, and complexity of a damaged microenvironment or a blood environment, retention and survival rates of the cells at a lesion site are low. Besides, in some cases, the directly injected cells may migrate from an injection site for differentiation into undesired types of cells. In such a way, the cells are prone to triggering a rejection, and thus are cleared by an immune system. This significantly limits effectiveness of cell therapy. Therefore, to further enhance therapeutic efficacy, a research on single-cell encapsulation has garnered widespread attention from scientists in the field of regenerative medicines. Hydrogel has a high water content, good biocompatibility, and good biodegradability. Its properties are closely similar to an extracellular matrix, and thus it is widely used for cell delivery. However, a current technology for preparing single-cell microspheres still faces numerous challenges. Most researchers prepare the single-cell microspheres using microfluidic devices. However, a microfluidic encapsulation system is costly and still faces the problems such as a low cell encapsulation rate and difficulty in preparing an ultra-thin gel layer. An adequately thin gel layer allows free passage of nutrients, oxygen, and water without limiting cell spreading, thereby promoting realization of cell functions. An excessive increase in microgel size poses limitations for applications such as high-throughput analysis and influences its pharmacokinetic behavior following implantation. In addition, researches have shown that intravenously injected ˜30 μm single-cell microspheres can accumulate in a pulmonary capillary bed of a mouse, whereas ˜50 μm microspheres are often physically intercepted, leading to accumulation in blood vessels of downstream tissues and posing a risk of tissue infarction. Second, a microfluidic strategy often inevitably introduces poorly biocompatible substances like oils and surfactants. The subsequent oil-removal step not only weakens cell viability but is also cumbersome, further limiting its widespread biomedical application.

Another common encapsulation strategy involves chemical modification, where cell surfaces are modified with exogenous substances, typically by embedding a respective group into a cell membrane through a chemical reaction. Although current chemically engineered cells show a good application prospect, they still face numerous challenges. For example, specific chemical reaction conditions may affect cell viability and offer poor controllability over thickness. Furthermore, exogenous substances modified on the cell surface are easily endocytosed by the cells, consequently compromising cell viability and delivery efficiency. As exogenous substances occupy sites on the cell surface, they can interfere binding receptors on the cell surfaces to specific ligands, which may impair the initiation of related signaling pathways and downstream functions. Additionally, this modification may elevate cellular immunogenicity, leading to recognition and clearance by immune cells. Mechanical properties of the cells may also be altered accordingly, resulting in weakened cell viability and alterations in other physicochemical properties. All these challenges pose major limitations to the biomedical applications of single-cell microspheres. Consequently, the aforementioned limitations motivate the development of preparation techniques for single-cell microspheres and led to its miniaturization. Encapsulating individual cells within a hydrogel layer at the nano-to micro-scale significantly increases the surface-to-volume ratio, thereby enhancing material exchange. This includes the efficient transport of nutrients and oxygen, while also facilitating the release of beneficial/therapeutic factors secreted by the cells to exert their intended functions. Therefore, developing a simple, versatile, and highly biocompatible innovative strategy for preparing single-cell microspheres to enhance their applications in the field of biomedical engineering is an urgent scientific challenge.

### SUMMARY

An objective of this section is to overview some aspects of examples of the present invention and to briefly introduce some of preferable examples. In the section as well as in the abstract and title of the present application, some simplifications or omissions may be made to avoid making the purpose of the section, the abstract and the title ambiguous, but such simplifications or omissions cannot be used to limit the scope of the present invention.

The present invention is provided in view of the above problems or the problems in the prior art.

Therefore, an objective of the present invention is to overcome the shortcomings in the prior art, and provide a method for preparing single-cell microspheres based on an oil-free aqueous two-phase system strategy.

In order to solve the above technical problems, the present invention provides the following technical solution: a method for preparing single-cell microspheres based on an oil-free aqueous two-phase system strategy, including:
resuspending cells in an upper solution, and slowly dripping the resuspended cells into a container containing a lower solution to form stable phase separation with the lower solution; and
curing, after the cells descend from the upper layer to the lower layer, a cell encapsulation layer to obtain the single-cell microspheres.

As a preferred solution of the method of the present invention, the upper solution is a protein-based solution or a hydrogel solution, having a concentration ranging from 0.001% to 2%.

As a preferred solution of the method of the present invention, the upper solution is one of the following: an alginate solution, a gelatin methacrylate anhydride solution, a hyaluronic acid methacrylate solution, a carboxymethyl cellulose solution, a matrigel solution, a sericin solution, a collagen solution, and a silk fibroin solution.

As a preferred solution of the method of the present invention, the lower solution is a polysaccharide aqueous solution or the protein-based solution, having a concentration ranging from 2% TO 20%.

As a preferred solution of the method of the present invention, the lower solution is one of the following: a dextran solution, a ficoll solution, a sucrose solution, an agarose solution, the sericin solution, the collagen solution, and the silk fibroin solution.

As a preferred solution of the method of the present invention, a method of curing a cell encapsulation layer is dropwise addition of the solution or other curing methods.

As a preferred solution of the method of the present invention, the dropwise addition of the solution is to add a calcium chloride solution or ferric chloride solution dropwise.

As a preferred solution of the method of the present invention, the other curing methods involve curing hydrogel by means of ultraviolet light or a temperature.

Another objective of the present invention is to overcome the shortcomings in the prior art and provide a single-cell microsphere prepared based on an oil-free aqueous two-phase system strategy.

As a preferred solution of the method of the present invention, an encapsulation rate of the single-cell microsphere is 98% or above.

The beneficial effects of the present invention are as follows:
(1) The present invention provides a method using highly biocompatible aqueous solutions, eliminating the need for oil and surfactants from conventional microfluidics. This significantly increases a cell survival rate, reaching 95% or above, while also simplifying the protocol by eliminating oil removal and reducing negative impacts on cell viability.
(2) The single-cell microspheres prepared by the present invention can not only achieve an encapsulation rate exceeding 98%, but also enable complete encapsulation.
(3) The present invention does not involve a specific chemical reaction but relies solely on a physical interaction. This can enhance the cell viability and enables indistinguishable encapsulation, thereby solving the problems such as occupied cell surface sites due to chemical encapsulation, an effect on binding the specific ligands to receptors, and improved immunogenicity.
(4) By adjusting the concentration of the solution, the present invention enables an controllable encapsulation thickness; and in this way, ultra-thin (1 μm to 5 μm) hydrogel-based single-cell microspheres that are difficult to achieve with the traditional microfluidic strategy are prepared.

(5) The preparation method, based on the oil-free aqueous two-phase system strategy, proposed by the present invention exhibits high throughput, excellent biocompatibility, excellent thickness controllability, and excellent operational simplicity.

### BRIEF DESCRIPTION OF DRAWINGS

To describe the technical solutions in examples of the present invention more clearly, the following briefly describes the accompanying drawings required for describing the examples. Apparently, the accompanying drawings in the following descriptions show merely some examples of the present invention, and those of ordinary skill in the art can derive other drawings from these drawings without any creative efforts. In the drawing:

FIG. 1 is a schematic flowchart of single-cell microspheres prepared based on an oil-free aqueous two-phase system strategy according to the present invention;

FIG. 2 shows phase separation of different solutions;

FIG. 3 shows microspheres encapsulating individual Hela cells, prepared using different solution combinations;

FIG. 4 shows single-cell microspheres encapsulating different types of cells, obtained utilizing stable two-phase separation;

FIG. 5 shows single-cell microspheres with varying encapsulation thicknesses, obtained by adjusting concentrations of alginate and dextran solutions; and

FIG. 6 shows a fluorescence image of h1299 cells from live/dead staining under the condition of 0.0125% alginate and 10% dextran.

### DETAILED DESCRIPTION OF THE EMBODIMENTS

To make the aforementioned objectives, features and advantages of the present invention more apparent and comprehensible, the specific examples of the present invention are described in detail below with reference to the examples of the specification.

Many specific details are set forth in the following description to facilitate a full understanding of the present invention, but the present invention can also be implemented in other ways different from those described here, and those skilled in the art can do similar promotions without departing from the connotation of the present invention. Therefore, the present invention is not limited by the specific examples disclosed below.

Secondly, the term “one example” or “examples” referred to here refers to specific features, structures, or features that may be included in at least one implementation of the present invention. The “in one example” appearing in different parts of the present specification does not necessarily refer to the same example, nor a separate or selective example that is mutually exclusive to other examples.

Materials and reagents used in the examples of the present invention: Zeiss confocal microscope (LSM880), MSHOT microscope (MF53-N), Alginate (AR grade, Sigma), carboxymethyl cellulose V (CMC V, Aladdin, JAD-C104983), Matrigel (corning, 356234), gelatin methacrylate (GelMA), Sericin, calcium chloride (AR grade, Damao Chemical), Dextran (Aladdin, D490149-750K), Ficoll (Sigma, F2637), Sucrose (Macklin, S818046), Gelatin (Sigma, G7041), Collagen (Macklin, C823250-100g), Silk fibroin (EFL-SF-001), and Agarose (S14003-10g).

### Example 1

A method for preparing single-cell microspheres based on an oil-free aqueous two-phase system strategy includes the following steps:
(1) Preparation of a 0.0125% alginate solution and a 10% dextran solution:
12.5 mg of an alginate solid was accurately weighed and then dissolved into 100 ml of a PBS solution to obtain 0.0125% alginate; and
10 g of a dextran solid was accurately weighed and then dissolved into 100 ml of a PBS solution to obtain 10% dextran.

To characterize successful cell encapsulation, fluorescent nanoparticles were added to the alginate solution to prepare a 0.5% fluorescent nanoparticle solution.
(2) Preparation of a calcium chloride solution:
a 1% calcium chloride solution was prepared by accurately weighing 1 g of calcium chloride and dissolving it into 100 ml of distilled water.
(3) Preparation of single-cell microspheres:
500 μL of a lower solution (the 10% dextran solution) was added to a suitable container in advance; then, Hela cells were resuspended in 500 μL of an upper solution (the 0.0125% alginate solution) mixed with 0.5% fluorescent nanoparticles; and a cell suspension was further slowly dripped into the container to form stable phase separation with the lower solution.

After the cells descended from the upper layer to the lower layer, the 1% calcium chloride solution was added dropwise to cure a cell encapsulation layer. Encapsulation was observed under a microscope, and a size of each microsphere was recorded.

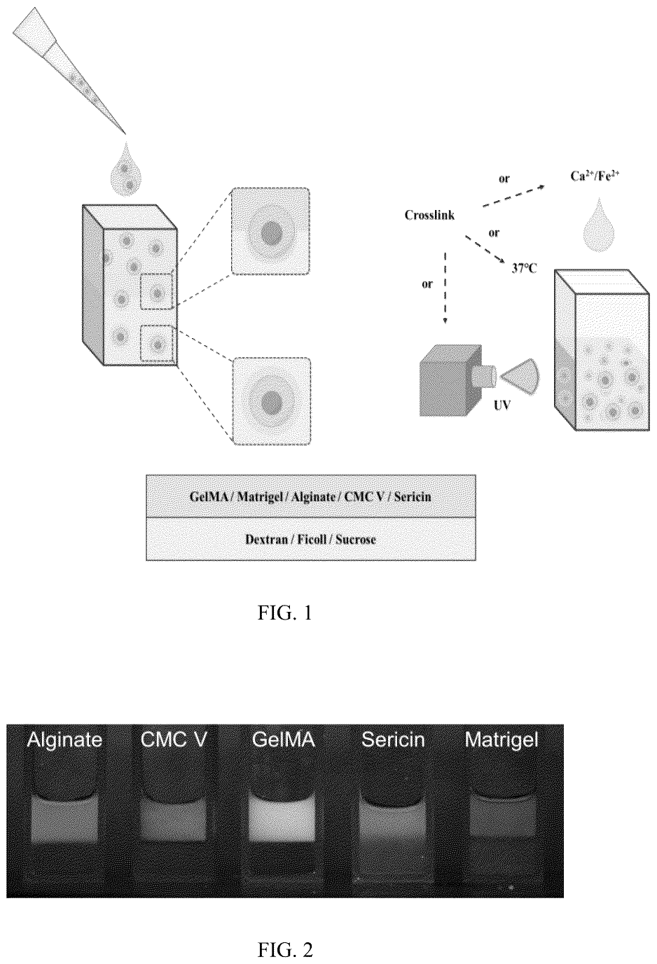

A schematic flowchart of single-cell microspheres prepared based on an oil-free aqueous two-phase system strategy is shown in FIG. 1.

### Example 2

A method for preparing single-cell microspheres based on an oil-free aqueous two-phase system strategy includes the following steps:
(1) Preparation of a 0.0125% carboxymethyl cellulose (CMC V) solution and a 10% dextran solution:
12.5 mg of a CMC V solid was accurately weighed and then dissolved into 100 ml of a 0.01 solution to obtain a 0.0125% CMC V solution; and
10 g of a dextran solid was accurately weighed and then dissolved into 100 ml of a PBS solution to obtain 10% dextran.

To characterize successful cell encapsulation, fluorescent nanoparticles were added to the CMC V solution to prepare a 0.5% fluorescent nanoparticle solution.
(2) Preparation of a ferric chloride solution:
a 1% ferric chloride solution was prepared by accurately weighing 1 g of ferric chloride and dissolving it into 100 ml of distilled water.
(3) Preparation of single-cell microspheres:
500 μL of a lower solution (the 10% dextran solution) was added to a suitable container in advance; then, Hela cells were resuspended in 500 μL of an upper solution (the 0.0125% CMC V solution) mixed with 0.5% fluorescent nanoparticles; and a cell suspension was further slowly dripped into the container to form stable phase separation with the lower solution.

After the cells descended from the upper layer to the lower layer, the 1% ferric chloride solution was added dropwise to cure a cell encapsulation layer. Encapsulation was observed under a microscope, and a size of each microsphere was recorded.

### Example 3

A method for preparing single-cell microspheres based on an oil-free aqueous two-phase system strategy includes the following steps:
(1) Preparation of a 0.0125% matrigel solution and a 10% dextran solution:
12.5 μL. of a matrigel solution was accurately weighed and then dissolved into 100 ml of a PBS solution to obtain the 0.0125% matrigel solution; and
10 g of a dextran solid was accurately weighed and then dissolved into 100 ml of a PBS solution to obtain 10% dextran.

To characterize successful cell encapsulation, fluorescent nanoparticles were added to the matrigel solution to prepare a 0.5% fluorescent nanoparticle solution.
(2) Preparation of single-cell microspheres:
500 μL of a lower solution (the 10% dextran solution) was added to a suitable container in advance; then Hela cells were resuspended in 500 μL of an upper solution (the 0.0125% matrigel solution) mixed with 0.5% fluorescent nanoparticles; and a cell suspension was further slowly dripped into the container to form stable phase separation with the lower solution.

After the cells descended from the upper layer to the lower layer, the solution was placed at 37° C. to cure a cell encapsulation layer. Encapsulation was observed under a microscope, and a size of each microsphere was recorded.

### Example 4

### (1) Synthesis of Gelatin Methacrylate Anhydride (GelMA) and Preparation of a GelMA Solution

Fish skin gelatin at a concentration of 10% was dissolved into phosphate-buffered saline (PBS) at 50° C.; then, methacrylic anhydride was gradually added dropwise using a syringe pump until a concentration of a resultant reached 4% (v/v); and the solution was stirred on a magnetic stirrer at 50° C. for 2 h to ensure homogeneity.

The GelMA was subjected to two dilutions and dialyzed with deionized water at 40° C. for 7 d, with the deionized water replaced every 12 h; the solution was filtered at 40° C. using a 0.22 μm sterile vacuum filtration system; the filtered solution was aliquoted in 25 ml portions, freezed at −80° C. for 1 d, and then lyophilized in a lyophilizer for 5 d to obtain a GelMA solid.

1 g of a GelMA solid was accurately weighed and then dissolved into 10 ml of a PBS solution to obtain the 10% GelMA solution.

To characterize successful cell encapsulation, the GelMA was cured via ultraviolet light in this experiment. Therefore, lithium phenyl(2,4,6-trimethylbenzoyl) phosphinate (LAP), as a photoinitiator, was added to the GelMA solution to prepare a GelMA solution containing 0.03% LAP.

### (2) Preparation of a 10% Dextran Solution

10 g of a dextran solid was accurately weighed and then dissolved into 100 ml of a PBS solution to obtain 10% dextran.

### (3) Preparation of Single-Cell Microspheres

500 μL of a lower solution (the dextran solution) was added to a suitable container in advance; then Hela cells were resuspended in 500 μL of the GelMA solution containing the 0.03% LAP; and a cell suspension was further slowly dripped into the container to form stable phase separation with the lower solution. After the cells descended from the upper layer to the lower layer, the solution was irradiated by an ultraviolet lamp (Shanggefei X9) for 30 s to cure a cell encapsulation layer. Encapsulation was observed under a microscope, and a size of each microsphere was recorded.

### Example 5

### (1) Synthesis of Sericin and Preparation of a Sericin Solution

Silkworm cocoons were cut into small pieces, which were washed with pure water three times, and aired; 50 g of the aired silkworm cocoons were weighed; 1 L of pure water was measured, to which 2.12 g of sodium carbonate (0.02 M) was added, and heated to boiling; the silkworm cocoons were put in the boiling water for boiling for 30 min, during which the silkworm cocoons were periodically scratched for separation; and after they were cooled to a room temperature, silk fibers were taken out, the remaining solution was centrifuged at 3500 rpm for 8 min at 4° C. to remove insoluble impurities.

A supernatant was taken and put into a dialysis bag (with MW being 6000-8000) for dialysis for 3 d, changing water four times daily; the dialyzed solution was centrifuged at 3500 rpm for 15 min at 4° C. to remove precipitated impurities; and the solution was divided into two portions: one portion was put in a dialysis bag and blown to dry in a fume hood using an electric fan until a desired concentration was achieved, and the other portion was frozen at −80° C., and then lyophilized in a lyophilizer for 3 d to obtain a Sericin solid.

1 g of the Sericin solid was accurately weighed and dissolved into 10 ml of a PBS solution to obtain the 10% Sericin solution. To characterize successful cell encapsulation, fluorescent nanoparticles were added to the solution to prepare a 0.5% fluorescent nanoparticle solution.

### (2) Preparation of a 10% Dextran Solution

10 g of a dextran solid was accurately weighed and then dissolved into 100 ml of a PBS solution to obtain 10% dextran.

### (3) Preparation of Single-Cell Microspheres

500 μL of a lower solution (the 10% dextran solution) was added to a suitable container in advance; then Hela cells were resuspended in 500 μL of an upper solution (the 10% Sericin solution) mixed with 0.5% fluorescent nanoparticles; and a cell suspension was further slowly dripped into the container to form stable phase separation with the lower solution.

After the cells descended from the upper layer to the lower layer, the solution was irradiated by green light with an intensity of 60 mW cm⁻² for 30 s to cure a cell encapsulation layer. Encapsulation was observed under a microscope, and a size of each microsphere was recorded.

Phase separation of the 0.0125% alginate solution, the CMC V solution, and the matrigel solution prepared in examples 1-5 with the 10% dextran solution, as well as phase separation of the 10% GelMA solution and the Sericin solution with the 10% dextran solution are shown in FIG. 2, in which stable phase separation can be observed.

Example 6

A method for preparing single-cell microspheres based on an oil-free aqueous two-phase system strategy includes the following steps:
(1) Preparation of a 0.0125% hyaluronic acid methacrylate (HAMA) solution and a 10% dextran solution:
12.5 mg of a HAMA solution was accurately weighed and then dissolved into 100 ml of a PBS solution to obtain the 0.0125% HAMA solution; and
10 g of a dextran solid was accurately weighed and then dissolved into 100 ml of a PBS solution to obtain 10% dextran.

To characterize successful cell encapsulation, fluorescent nanoparticles were added to the matrigel solution to prepare a 0.5% fluorescent nanoparticle solution.

### (2) Preparation of Single-Cell Microspheres

500 μL of a lower solution (the 10% dextran solution) was added to a suitable container in advance; then Hela cells were resuspended in 500 μL of an upper solution (the 0.0125% HAMA solution) mixed with 0.5% fluorescent nanoparticles; and a cell suspension was further slowly dripped into the container to form stable phase separation with the lower solution.

After the cells descended from the upper layer to the lower layer, the solution was irradiated by an ultraviolet lamp (Shanggefei X9) for 30 s to cure a cell encapsulation layer. Encapsulation was observed under a microscope, and a size of each microsphere was recorded.

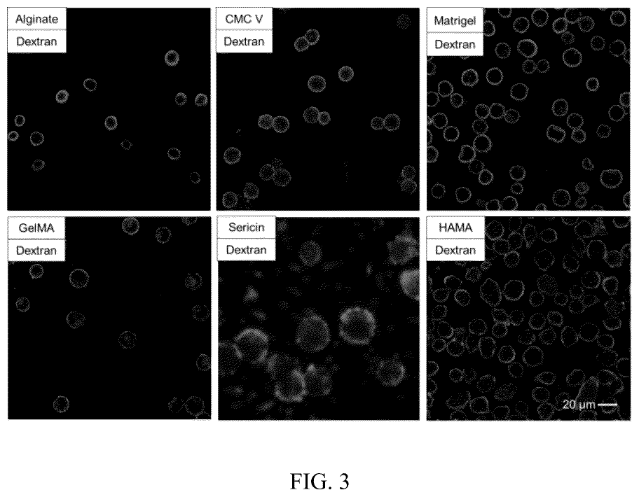

Microspheres encapsulating individual Hela cells prepared in examples 1-6 using combinations of the alginate solution, the CMC V solution, the matrigel solution, the GelMA solution, the Sericin solution, and the HAMA solution with the dextran solution respectively are shown in FIG. 3, in which it can be observed that the cells are completely encapsulated, demonstrating a very high single-cell encapsulation rate.

### Example 7

This example is different from example 1 in that: in step (4), the encapsulated cells are respectively the Hela cells, human non-small cell lung cancer cells (A549), rat myocardial cells (H9C2), mouse myoblasts (C2C12), human non-small cell lung cancer cells (h1299), and mesenchymal stem cells (MSC). All other steps are the same as in example 1.

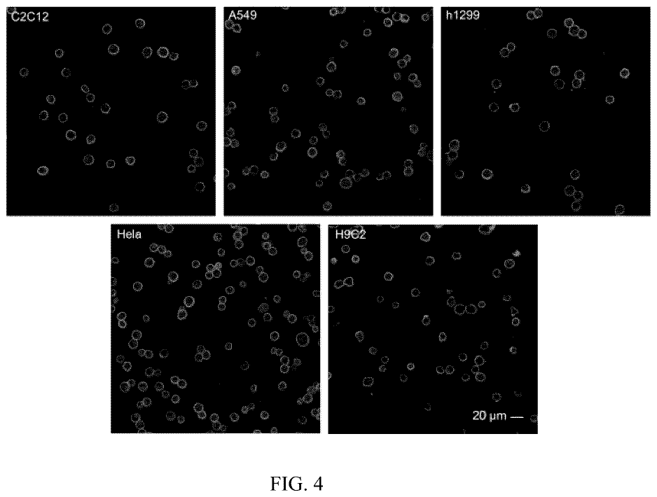

FIG. 4 shows single-cell microspheres encapsulating different types of cells, obtained utilizing stable two-phase separation. It can be observed that various types of cells can be encapsulated.

### Example 8

This example is different from example 1 in that: the alginate solution is prepared with varying concentrations of 0.125%, 0.25%, and 0.5%. All other steps are the same as in example 1.

### Example 9

This example is different from example 1 in that: the dextran solution is prepared with varying concentrations of 2.5%, 5%, and 7.5%. All other steps are the same as in example 1.

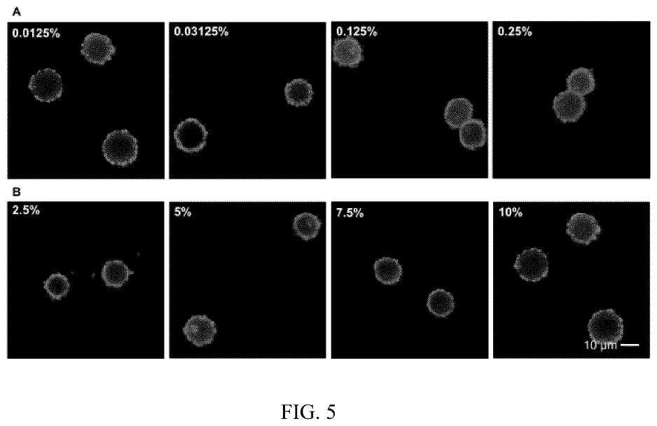

FIG. 5 shows the single-cell microspheres with different encapsulation thicknesses obtained in examples 8 and 9 by adjusting the concentrations of the alginate and dextran solutions. It can be observed that through a permutation and combination experiment with varying concentrations, a thickness of a gel layer is controllable to below 5 μm.

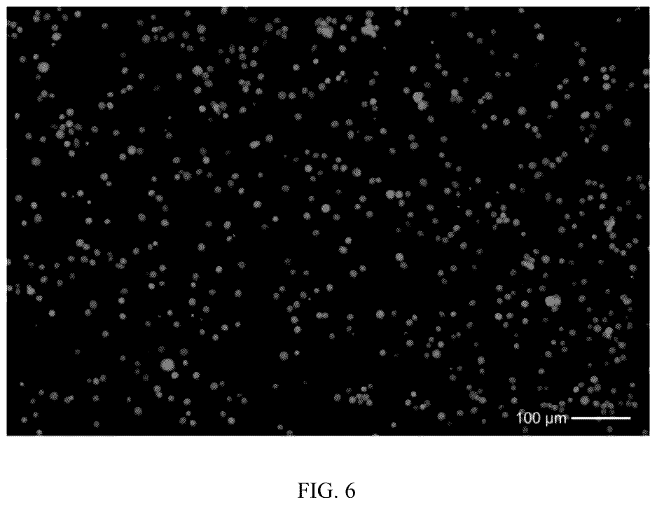

FIG. 6 shows a fluorescence image of h1299 cells from live/dead staining under the conditions of 0.0125% alginate and 10% dextran showing excellent cell viability of over 95%.

### Comparative Example 1

This comparative example is different from example 1 in that: the alginate solution is prepared with the concentration being 3%. All other steps are the same as in example 1. A result has shown that the cells remained suspended in the upper solution and failed to descend to the lower layer.

### Comparative Example 2

This comparative example is different from example 1 in that: the dextran solution is prepared with the concentration being 1%. All other steps are the same as in example 1. A result has shown that low concentrations of alginate and dextran were difficult to form a stable interface, preventing the cells from being encapsulated.

### Comparative Example 3

This comparative example is different from example 1 in that: the alginate solution is replaced with 2% polyvinyl alcohol (PVA). All other steps are the same as in example 1. A result has shown that the cells were unable to be encapsulated.

### Comparative Example 4

This comparative example is different from example 4 in that: the GelMA solution has a concentration of 20%. All other steps are the same as in example 4. A result has shown unstable phase separation, which prevented the cells from being encapsulated.

### Comparative Example 5

This comparative example is different from example 1 in preparing the single-cell microspheres using a traditional microfluidic technology, including the following steps:
cells were mixed into a 0.0125% alginate solution to serve as a dispersed phase, and a resultant was injected into a microfluidic chip;
fluorinated oil (Fluorinert FC-40, Sigma Aldrich) containing a 2% w/w fluorosurfactant (RAN BioTechnologies) was used as a continuous phase; and
an oil phase was used to shear a hydrogel solution, to obtain single-cell microdroplets, which were then collected in a 1% calcium chloride solution to form the single-cell microspheres.

A result has shown that the single-cell microspheres obtained using the microfluidic technology exhibited an encapsulation rate of 40% and a cell survival rate of 75%, both of which were significantly lower than those achieved by the oil-free aqueous two-phase system strategy proposed in the present invention.

The present invention utilizes a highly biocompatible aqueous solution, without use of oil and a surfactant required in a traditional microfluidic strategy. This significantly increases a cell survival rate, reaching 95% or above, simplifies experimental operations by eliminating the oil removal step, and also weakens adverse effects on cell viability. The single-cell microspheres prepared in the present invention can not only achieve an encapsulation rate exceeding 98%, but also enable complete encapsulation.

The present invention does not involve a specific chemical reaction but relies solely on a physical interaction. This can enhance the cell viability and enables indistinguishable encapsulation, thereby solving the problems such as occupied cell surface sites due to chemical encapsulation, an effect on binding specific ligands to receptors, and improved immunogenicity. By adjusting the concentration of the solution, the present invention enables precise control over the encapsulation thickness, facilitating preparation of ultra-thin (1 μm to 5 μm) hydrogel-based single-cell microspheres that are difficult to achieve with the traditional microfluidic strategy. The preparation method, based on the oil-free aqueous two-phase system strategy, proposed by the present invention exhibits high throughput, excellent biocompatibility, excellent thickness controllability, and excellent operational simplicity.

It should be noted that the above examples are merely used to describe, but not to limit, the technical solutions of the present invention. Although the present invention is described in detail with reference to the preferred examples, those of ordinary skill in the art should understand that the technical solutions of the present invention can be modified or equivalently replaced without departing from the spirit and scope of the technical solutions of the present invention, and should fall within the scope of the present invention.

What is claimed is:

1. A method for preparing single-cell microspheres based on an oil-free aqueous two-phase system strategy, comprising:
resuspending cells in an upper solution, and slowly dripping the resuspended cells into a container containing a lower solution to form stable phase separation with the lower solution; and
curing, after the cells descend from the upper layer to the lower layer, a cell encapsulation layer to obtain the single-cell microspheres.

2. The method according to claim 1, wherein the upper solution is a protein-based solution or a hydrogel solution, having a concentration ranging from 0.001% to 2%.

3. The method according to claim 1, wherein the upper solution is one of the following: an alginate solution, a gelatin methacrylate anhydride solution, a carboxymethyl cellulose solution, a matrigel solution, a sericin solution, a collagen solution, a silk fibroin solution, and hyaluronic acid methacrylate.

4. The method according to claim 1, wherein the lower solution is a polysaccharide aqueous solution or the protein-based solution, having a concentration ranging from 2% to 20%.

5. The method according to claim 1, wherein the lower solution is one of the following: a dextran solution, a ficoll solution, a sucrose solution, an agarose solution, the sericin solution, the collagen solution, and the silk fibroin solution.

6. The method according to claim 1, wherein a method of curing a cell encapsulation layer is dropwise addition of the solution or other curing methods.

7. The method according to claim 6, wherein the dropwise addition of the solution is to add a calcium chloride solution or ferric chloride solution dropwise.

8. The method according to claim 6, wherein the other curing methods involve curing hydrogel by means of ultraviolet light or a temperature.

9. A single-cell microsphere prepared by the method according to claim 1.

10. The single-cell microsphere according to claim 9, wherein an encapsulation rate of the single-cell microsphere is 98% or above.
